(19) United States
(12) Patent Application Publication
(10) Pub. No.: US 2026/0076843 A1
(43) Pub. Date: Mar. 19, 2026

(54) CARBON FIBER CORE, AND PREPARATION METHOD AND APPLICATION THEREOF

(71) Applicants: BJBEST maternal and child products Co., LTD, Beijing (CN); JAYFEN HEALTH (MALAYSIA) SDN.BHD., Klang (MY)

(72) Inventors: Qiuhong Li, Beijing (CN); Jie Zhang, Beijing (CN); Bo Li, Beijing (CN); Baoling Hu, Beijing (CN); Xiaogang Qu, Beijing (CN)

(21) Appl. No.: 19/396,399

(22) Filed: Nov. 21, 2025

Related U.S. Application Data
(63) Continuation of application No. PCT/CN2024/083366, filed on Mar. 22, 2024.

(30) Foreign Application Priority Data
May 22, 2023 (CN) 202310583146.2

(51) Int. Cl. A61F 13/537; A61F 13/15; A61F 13/53; A61F 13/534

(52) U.S. Cl. CPC A61F 13/53747; A61F 2013/1591; A61F 2013/15967; A61F 2013/530131; A61F 2013/5349

(57) ABSTRACT

The invention discloses a carbon fiber core, which includes a carbon fiber core body. The carbon fiber core body comprises, from top to bottom, a diversion layer, an adhesive layer I, a wrapping layer I, an adhesive layer II, a carbon fiber layer, a fluff pulp layer, a water-absorbent resin layer, an adhesive layer III, and a wrapping layer II. The wrapping layers and the core layer are provided with liquid-permeable and air-permeable holes. On the premise of having a reasonable structure and ensuring absorption performance and anti-reflux performance, the present invention also has excellent deodorizing performance and bacteriostatic performance, and can be applied to incontinence pads and sanitary napkins. In addition, the preparation process of the present invention can realize mass production of the core and applied products, solving the problems of poor production quality and slow production speed existing in the previous sanitary products made of activated carbon.

### CROSS-REFERENCE TO RELATED APPLICATIONS

The present application is a continuation application of PCT application No. PCT/CN2024/083366 filed on Mar. 22, 2024, which claims the benefit of Chinese Patent Application No. 202310583146.2 filed on May 22, 2023. The contents of all of the aforementioned applications are incorporated by reference herein in their entirety.

### TECHNICAL FIELD

The present disclosure relates to the field of disposable hygiene products, especially relates to a carbon fiber core, its preparation method and application.

### BACKGROUND

The main performance characteristics of conventional personal sanitary products focus on leak resistance, comfortable use, convenience, dryness, and lightness. As people's requirements for the quality of life gradually increase, and there are more and more people with sensitive skin, in addition to the above basic performance requirements, the deodorizing performance and bacteriostatic performance of sanitary products have increasingly become the focus of customers' attention.

In recent years, the sanitary product industry has also seen a large number of technical researches and patent achievements regarding the bacteriostatic and deodorizing functions of sanitary napkins and incontinence products. However, most of these functions are achieved by adding chemical bacteriostatic ingredients and odor masking ingredients. At present, some products also try to use activated carbon or add bacteriostatic agents to obtain deodorizing and bacteriostatic functions for application in sanitary products, but there are problems such as color fading of activated carbon, which affects the user experience, and poor bacteriostatic and deodorizing effects.

Since the performance of the core is the key factor affecting the performance of adult incontinence pad products, the present invention has developed a new functional core with both deodorizing and bacteriostatic functions. It not only solves the deodorizing and bacteriostatic problems of incontinence pads and sanitary napkins, but also enables good production process control, realizes continuous production, and avoids pollution problems.

### SUMMARY OF THE INVENTION

To overcome the shortcomings referred in the prior art, the present invention provides a carbon fiber core, its preparation method and application. On the premise of having a reasonable structure and ensuring absorption performance and anti-reflux performance, the functional carbon fiber core of the present invention also has excellent deodorizing performance and bacteriostatic performance, and can be applied to incontinence pads and sanitary napkins.

The technical solution adopted by the present invention to solve its technical problems is as follows:

The carbon fiber core includes a carbon fiber core body;

The carbon fiber core body comprises, from top to bottom, a diversion layer, an adhesive layer I, a wrapping layer I, an adhesive layer II, a carbon fiber layer, a fluff pulp layer, a water-absorbent resin layer, an adhesive layer III, and a wrapping layer II. The wrapping layer I, the wrapping layer II, and the core layer between them are integrally provided with a plurality of liquid-permeable and air-permeable holes.

As an improvement of the above technical solution, the mass ratio of each component of the carbon fiber core body is:
diversion layer:adhesive layer I:wrapping layer I:adhesive layer II:carbon fiber layer:fluff pulp layer:water-absorbent resin layer:adhesive layer III:wrapping layer II=10-80%:0.5-5.0%:2-40%:0.5-5.0%:2-90%:0-95%:0-25%:0.5-5.0%:5-60%.

As an improvement of the above technical solution, the diversion layer is made of bicomponent fibers through a hot-air process.

As an improvement of the above technical solution, the diversion layer is glued to the wrapping layer I through the adhesive layer I.

As an improvement of the above technical solution, the adhesive layer I, adhesive layer II, and adhesive layer III are made of the same type of petroleum-based hot-melt pressure-sensitive adhesive, polyolefin-based hot-melt non-pressure-sensitive adhesive, or water-based EVA (Ethylene-Vinyl Acetate Copolymer) adhesive.

As an improvement of the above technical solution, the carbon fiber layer, the fluff pulp layer, and the water-absorbent resin layer are uniformly blended and laid flat between the wrapping layer I and the wrapping layer II.

As an improvement of the above technical solution, the carbon fiber layer is made of activated carbon fiber material with an iodine adsorption value of 800-1600 mg/g, the fiber length is between 0.2-30 mm, and the addition amount is between 2-30 g/m².

As an improvement of the above technical solution, the fluff pulp layer is made of fully processed, semi-processed, or unprocessed fluff pulp.

As an improvement of the above technical solution, the water-absorbent resin is sodium polyacrylate with a water absorption rate>5 and an absorption rate of 0.9% normal saline>20, and its particle size is between 0.5-2 mm.

As an improvement of the above technical solution, the liquid-permeable and air-permeable holes are semi-penetrating circular dots, square or diamond-shaped embossments, and the diameter of the pressing dots or embossments ranges from 0.5-60 mm.

Another objective of the present invention is to provide a method for preparing the above carbon fiber core, which includes:
S1: Preparing the diversion layer

The diversion layer is made of bicomponent fibers through a hot-air process. The Bicomponent fibers are selected from any two of the following materials: PP, PE, PET, PLA, and PBAT. The grammage ranges from 10-60 gsm, and the fiber fineness ranges from 6-12 D.
S2: Preparing the wrapping layer I and wrapping layer II
   The wrapping layer I and wrapping layer II are made of ordinary toilet paper, wet-strength toilet paper, or non-woven fabric, with a grammage ranging from 10-40 gsm;
   Among them, the wrapping layer I and wrapping layer II can be made of the same material or different materials;
   The toilet paper is made of wood pulp or bamboo pulp, and the non-woven fabric is mainly made of PE or PP;
   When the wrapping layer II is made of ordinary toilet paper or wet-strength toilet paper, the grammage ranges from 10-20 gsm.
S3: Obtaining adhesive layers based on the adhesive

The adhesive layer I, adhesive layer II, and adhesive layer III are made of the same type of petroleum-based hot-melt pressure-sensitive adhesive, polyolefin-based hot-melt non-pressure-sensitive adhesive, or water-based EVA adhesive.
S4: Gluing I

Through a spiral or spray sizing process, the diversion layer and the wrapping layer I are glued together through the adhesive layer I.
S5: Preparing the deodorizing and bacteriostatic layer
   The carbon fiber layer is made of activated carbon fiber material with an iodine adsorption value of 800-1600 mg/g, the fiber length is controlled between 0.2-30 mm, and the addition amount is controlled between 2-30 g/m²;
   The fluff pulp layer is made of fully processed, semi-processed, or unprocessed fluff pulp, and the moisture content is controlled below <10%;
   The water-absorbent resin is sodium polyacrylate with a water absorption rate>5 and an absorption rate of 0.9% normal saline>20, and its particle size is between 0.5-2 mm;
The carbon fiber layer, the fluff pulp layer, and the water-absorbent resin layer are uniformly blended to obtain the deodorizing and bacteriostatic layer and laid flat between the wrapping layer I and the wrapping layer II;
The usage ratio of carbon fiber, fluff pulp, and water-absorbent resin in the deodorizing and bacteriostatic layer is 30-100%:0-50%:0-20%.
S6: Gluing II
   Through a spiral sizing process or a spray sizing process, the wrapping layer I and the deodorizing and bacteriostatic layer is glued through the adhesive layer II, and the deodorizing and bacteriostatic layer is glued through the adhesive layer III;
   The operating temperature is between 100-180° C., and the viscosity during operation is between 1700 cps-15000 cps.
S7: Making the liquid-permeable and air-permeable holes
   The whole composed of the wrapping layer I, adhesive layer II, carbon fiber layer, fluff pulp layer, water-absorbent resin layer, adhesive layer III, and wrapping layer II is pressed by a pressure roller to form a plurality of liquid-permeable and air-permeable holes;
   The liquid-permeable and air-permeable holes can be semi-penetrating circular dots, square or diamond-shaped embossments, and the diameter of the pressing dots or embossments ranges from 0.5-60 mm.

Another objective of the present invention is to provide an application of the carbon fiber core in disposable care sanitary products.

As an improvement of the above technical solution, the disposable care sanitary products include incontinence pads, urine pads, diapers, panty liners, or sanitary napkins.

As an improvement of the above technical solution, the disposable care sanitary products need to have good infiltration performance and anti-reflux performance. Among them, the infiltration index of sanitary napkins needs to be <60 s, and the anti-reflux performance needs to be <1 g; the infiltration index of incontinence products needs to be <15 s each time, and the anti-reflux amount needs to be <2 g.

The beneficial effects brought by the present invention are:

The carbon fiber core and its formula provided by the present invention have bacteriostatic and deodorizing functions and effects, enabling the product to effectively inhibit bacteria and odors in excreta.

The carbon fiber core structure provided by the present invention reasonably integrates the core with bacteriostatic and deodorizing effects, so that when the core is applied to products, it can also achieve good infiltration and dryness performance.

The carbon fiber core product can be perfectly applied to functional sanitary napkins, incontinence pads and other products, solving the problem of excreta odor for women during menstruation and incontinent people.

Based on the preparation process of the carbon fiber core of the present invention, mass production of the core and applied products can be realized, which solves the problems of poor production quality and slow production speed existing in the previous sanitary products made of activated carbon.

### BRIEF DESCRIPTION OF FIGURES

The present invention will be further described in detail below with reference to the accompanying drawings and specific embodiments:

FIG. 1 is a schematic diagram of the structure of the carbon fiber core in Example 1;

FIG. 2 is a partial schematic diagram of the carbon fiber core in Example 1.

### DETAILED DESCRIPTION

The technical solutions in the embodiments of the present invention will be clearly and completely described below. Obviously, the described embodiments are only a part of the embodiments of the present invention, rather than all the embodiments. Based on the embodiments of the present invention, all other embodiments obtained by those of ordinary skill in the art without making creative work shall fall within the protection scope of the present invention.

### Example 1

The carbon fiber core includes a carbon fiber core body;

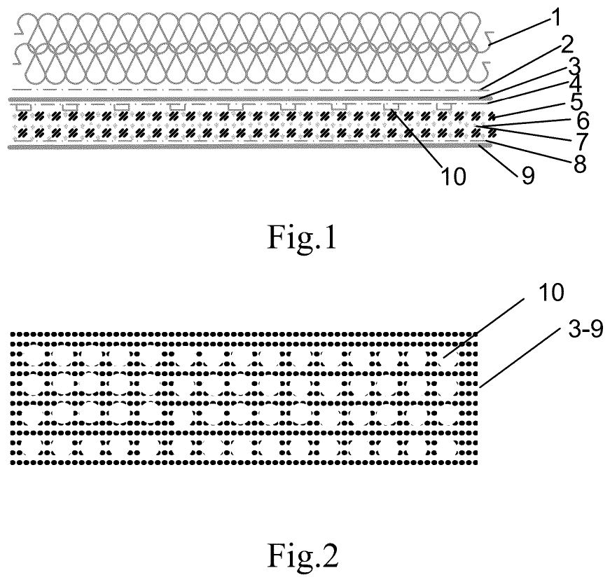

With reference to FIGS. 1-2, the carbon fiber core body comprises, from top to bottom in sequence, a diversion layer 1, an adhesive layer I 2, a wrapping layer I 3, an adhesive layer II 4, a carbon fiber layer 5, a fluff pulp layer 6, a water-absorbent resin layer 7, an adhesive layer III 8, and a wrapping layer II 9. The wrapping layer I 3, the wrapping layer II 9, and the core layer between them are integrally provided with a plurality of liquid-permeable and air-permeable holes 10.

As an improvement of the above embodiment, the mass ratio of each component of the carbon fiber core body is:
diversion layer 1:Adhesive layer I 2:Wrapping layer I 3:Adhesive layer II 4:Carbon fiber layer 5:Fluff pulp layer 6:Water-absorbent resin layer 7:Adhesive layer III 8:Wrapping layer II 9=10-80%: 0.5-5.0%: 2-40%: 0.5-5.0%: 2-90%: 0-95%: 0-25%: 0.5-5.0%: 5-60%.

As an improvement of the above embodiment, the diversion layer 1 is made of bicomponent fibers through a hot-air process, and its material is selected from any two of PP, PET, PE, PLA, and PBAT.

As an improvement of the above embodiment, the diversion layer 1 is glued to the wrapping layer I 3 through the adhesive layer I 2.

As an improvement of the above embodiment, the adhesive layer I 2, adhesive layer II 4, and adhesive layer III 8 are made of the same type of petroleum-based hot-melt pressure-sensitive adhesive, polyolefin-based hot-melt non-pressure-sensitive adhesive, or water-based EVA adhesive, such as the adhesive of Focus hoemelt T137.

As an improvement of the above embodiment, the carbon fiber layer 5, the fluff pulp layer 6, and the water-absorbent resin layer 7 are uniformly blended and laid flat between the wrapping layer I 3 and the wrapping layer II 9, so that the fluff pulp and carbon fiber material are uniformly mixed and laid flat, and the water-absorbent polymer is uniformly dispersed in the mixture of fluff pulp and carbon fiber.

As an improvement of the above embodiment, the carbon fiber layer 5 is made of activated carbon fiber material with an iodine adsorption value of 800-1600 mg/g, the fiber length is controlled between 0.2-30 mm, and the addition amount is controlled between 2-30 g/m².

As an improvement of the above embodiment, the fluff pulp layer 6 is made of fully processed, semi-processed, or unprocessed fluff pulp.

As an improvement of the above embodiment, the water-absorbent resin is an sodium polyacrylate with a water absorption rate>5 and an absorption rate of 0.9% normal saline>20, and its particle size is between 0.5-2 mm.

As an improvement of the above embodiment, the liquid-permeable and air-permeable holes 10 are semi-penetrating circular dots, square or diamond-shaped embossments, and the diameter of the pressing dots or embossments ranges from 0.5-60 mm.

### Example 2

A method for preparing the carbon fiber core described in Example 1, which includes:
S1: Preparing the diversion layer 1

The diversion layer 1 is made of bicomponent fibers through a hot-air process. Its grammage ranges from 10-60 gsm, the fiber fineness ranges from 6-12 D, and its material is any two types of fibers selected from PP, PET, PE, PLA, and PBAT.
S2: Preparing the wrapping layer I 3 and wrapping layer II 9
   The wrapping layer I 3 and wrapping layer II 9 are made of ordinary toilet paper, wet-strength toilet paper, or non-woven fabric, with a grammage ranging from 10-40 gsm;
   Among them, the wrapping layer I 3 and wrapping layer II 9 can be made of the same material or different materials;
   The toilet paper can be made of wood pulp or bamboo pulp, and the non-woven fabric is mainly made of PE or PP;
   When the wrapping layer II 9 is made of ordinary toilet paper or wet-strength toilet paper, the grammage ranges from 10-20 gsm.
S3: Obtaing the adhesive layers based on the adhesive

The adhesive layer I 2, adhesive layer II 4, and adhesive layer III 8 can be made of the same type of petroleum-based hot-melt pressure-sensitive adhesive, polyolefin-based hot-melt non-pressure-sensitive adhesive, or water-based EVA adhesive.
S4: Gluing I

Through a spiral or spray sizing process, the diversion layer 1 and the wrapping layer I 3 are glued together through the adhesive layer I 2.
S5: Preparing the deodorizing and bacteriostatic layer
   The carbon fiber layer 5 is made of activated carbon fiber material with an iodine adsorption value of 800-1600 mg/g, the fiber length is between 0.2-30 mm, and the addition amount is between 2-30 g/m²;
   The fluff pulp layer 6 is made of fully processed, semi-processed, or unprocessed fluff pulp, and the moisture content is controlled below<10%;
   The water-absorbent resin is sodium polyacrylate with a water absorption rate>5 and an absorption rate of 0.9% normal saline>20, and its particle size is between 0.5-2 mm;
   The carbon fiber layer 5, the fluff pulp layer 6, and the water-absorbent resin layer 7 are uniformly blended to obtain the deodorizing and bacteriostatic layer and laid flat between the wrapping layer I 3 and the wrapping layer II 9, and great attention should be paid to the flat laying effect;
   The usage ratio of carbon fiber, fluff pulp, and water-absorbent resin in the deodorizing and bacteriostatic layer is 30-100%:0-50%:0-20%;
   The deodorizing and bacteriostatic layer can achieve deodorizing effect and bacteriostatic effect. It has been verified that the reduction rate of the odor concentration of indole, hydrogen sulfide, acetic acid, methyl mercaptan, and ammonia gas is all greater than 70%, and the bacteriostatic rate against Escherichia coli, Staphylococcus aureus, and Candida albicans is greater than 70%.
S6: Gluing II
   Through a spiral or spray sizing process, the wrapping layer I 3 and the deodorizing and bacteriostatic layer, and the deodorizing and bacteriostatic layer and the wrapping layer II 9 are respectively glued through the adhesive layer II 4 and the adhesive layer III 8, and great attention should be paid to the adhesion performance to the carbon fiber material;
   The operating temperature is between 100-180° C., and the viscosity during operation is between 1700 cps-15000 cps.
S7: Making the liquid-permeable and air-permeable holes 10
   The core layer composed of the wrapping layer I 3, adhesive layer II 4, carbon fiber layer 5, fluff pulp layer 6, water-absorbent resin layer 7, adhesive layer III 8, and wrapping layer II 9 is pressed by a pressure roller to form a plurality of liquid-permeable and air-permeable holes 10;
   The liquid-permeable and air-permeable holes 10 can be semi-penetrating circular dots, square or diamond-shaped embossments;
   The diameter of the pressing dots is determined according to the width of the selected material, and the diameter of the pressing dots or embossments ranges from 0.5-60 mm.

### Example 3

Application of the carbon fiber core prepared by the preparation method described in Example 2 and as described in Example 1 in disposable care sanitary products.

The carbon fiber core can be used to produce disposable care sanitary products including, but not limited to, incontinence pads, urine pads, diapers, panty liners, or sanitary napkins.

In addition to having bacteriostatic and deodorizing performance, the disposable care sanitary products also need to have good infiltration performance and anti-reflux performance. Among them, the infiltration index of sanitary napkins needs to be <60 s, and the anti-reflux performance needs to be <1 g; the infiltration index of incontinence products needs to be <15 s each time, and the performance amount needs to be <2 g.

It should be noted that the above are only the preferred embodiments of the present invention and are not intended to limit the present invention. Although the present invention has been described in detail with reference to the foregoing embodiments, for those skilled in the art, they can still modify the technical solutions described in the foregoing embodiments or replace some of the technical features with equivalents. Any modification, equivalent replacement, improvement, etc. made within the spirit and principle of the present invention shall be included in the protection scope of the present invention.

1. A carbon fiber core, comprising a carbon fiber core body, wherein the carbon fiber core body comprising, from top to bottom, a diversion layer, an adhesive layer I, a wrapping layer I, an adhesive layer II, a carbon fiber layer, a fluff pulp layer, a water-absorbent resin layer, an adhesive layer III, and a wrapping layer II;
the wrapping layer I, the wrapping layer II, and the core layer between them are integrally provided with a plurality of liquid-permeable and air-permeable holes;
the mass ratio of each component of the carbon fiber core body is: diversion layer:adhesive layer I:wrapping layer I:adhesive layer II:carbon fiber layer:fluff pulp layer:water-absorbent resin layer:adhesive layer III:wrapping layer II=10-80%:0.5-5.0%:2-40%:0.5-5.0%:2-90%:0-95%:0-25%:0.5-5.0%:5-60%.

2. The carbon fiber core according to claim 1, wherein the diversion layer is made of bicomponent fibers through a hot-air process; the diversion layer is glued to the wrapping layer I through the adhesive layer I.

3. The carbon fiber core according to claim 1, wherein the adhesive layer I, adhesive layer II, and adhesive layer III are made of the same type of petroleum-based hot-melt pressure-sensitive adhesive, polyolefin-based hot-melt non-pressure-sensitive adhesive, or water-based EVA adhesive.

4. The carbon fiber core according to claim 1, wherein the carbon fiber layer, the fluff pulp layer, and the water-absorbent resin layer are uniformly blended and laid flat between the wrapping layer I and the wrapping layer II;
the carbon fiber layer is made of activated carbon fiber material with an iodine adsorption value of 800-1600 mg/g, a fiber length is between 0.2-30 mm, and an addition amount is between 2-30 g/m²;
the fluff pulp layer is made of fully processed, semi-processed, or unprocessed fluff pulp;
the water-absorbent resin is sodium polyacrylate with a water absorption rate>5 and an absorption rate of 0.9% normal saline>20, and its particle size is between 0.5-2 mm.

5. The carbon fiber core according to claim 1, wherein the liquid-permeable and air-permeable holes are semi-penetrating circular dots, square or diamond-shaped embossments, and the diameter of the pressing dots or embossments ranges from 0.5-60 mm.

6. A method for preparing the carbon fiber core according to claim 1, comprising:
S1: preparing the diversion layer
the diversion layer is made of bicomponent fibers through a hot-air process,
the bicomponent fibers are selected from any two of the following materials: PP, PE, PET, PLA, and PBAT, a grammage ranges from 10-60 gsm, and a fiber fineness ranges from 6-12 D;
S2: preparing the wrapping layer I and wrapping layer II
the wrapping layer I and wrapping layer II are made of ordinary toilet paper, wet-strength toilet paper, or non-woven fabric, with a grammage ranging from 10-40 gsm;
wherein, the wrapping layer I and wrapping layer II can be made of the same material or different materials; when the wrapping layer II is made of ordinary toilet paper or wet-strength toilet paper, the grammage ranges from 10-20 gsm;
S3: obtaining adhesive layers based on the adhesive
the adhesive layer I, adhesive layer II, and adhesive layer III are made of the same type of petroleum-based hot-melt pressure-sensitive adhesive, polyolefin-based hot-melt non-pressure-sensitive adhesive, or water-based EVA adhesive;
S4: gluing I
through a spiral or spray sizing process, the diversion layer and the wrapping layer I are glued together through the adhesive layer I;
S5: preparing the deodorizing and bacteriostatic layer
the carbon fiber layer is made of activated carbon fiber material with an iodine adsorption value of 800-1600 mg/g, a fiber length is between 0.2-30 mm, and an addition amount is between 2-30 g/m²;
the fluff pulp layer is made of fully processed, semi-processed, or unprocessed fluff pulp, and the moisture content is <10%;
the water-absorbent resin is sodium polyacrylate with a water absorption rate>5 and an absorption rate of 0.9% normal saline>20, and its particle size is between 0.5-2 mm;
the carbon fiber layer, the fluff pulp layer, and the water-absorbent resin layer are uniformly blended to obtain the deodorizing and bacteriostatic layer and laid flat between the wrapping layer I and the wrapping layer II;
S6: gluing II
through a spiral sizing process or a spray sizing process, the wrapping layer I and the deodorizing and bacteriostatic layer, and the deodorizing and bacteriostatic layer is glued through the adhesive layer II, and the deodorizing and bacteriostatic layer is glued through the adhesive layer III;
a operating temperature is between 100-180° C, and a viscosity during operation is between 1700 cps-15000 cps; and
S7: making the liquid-permeable and air-permeable holes
a whole composed of the wrapping layer I, adhesive layer II, carbon fiber layer, fluff pulp layer, water-absorbent resin layer, adhesive layer III, and wrapping layer II is pressed by a pressure roller to form a plurality of liquid-permeable and air-permeable holes;
the liquid-permeable and air-permeable holes are semi-penetrating circular dots, square or diamond-shaped embossments, and the diameter of the pressing dots or embossments ranges from 0.5-60 mm.

7. Application of the carbon fiber core according to claim 1 in disposable care sanitary products, wherein taking the carbon fiber core to prepare the disposable care sanitary products.

8. The application according to claim 7, wherein the disposable care sanitary products include incontinence pads, urine pads, urine diapers, panty liners, or sanitary napkins.

9. The application according to claim 7, wherein the disposable care sanitary products have the following good infiltration performance and anti-reflux performance, the infiltration index of sanitary napkins<60 s, and the anti-reflux performance<1 g; the infiltration index of incontinence products<15 s each time, and the performance amount<2 g.
